(19) United States
(12) Patent Application Publication
(10) Pub. No.: US 2024/0199637 A1
(43) Pub. Date: Jun. 20, 2024

(54) IMPROVED PROCESS FOR THE PREPARATION OF 7-(MORPHOLINYL)-2-(N-PIPERAZINYL)METHYLTHIENO[2, 3-C]PYRIDINE DERIVATIVES

(71) Applicant: NATCO PHARMA LIMITED, Hyderabad (IN)

(72) Inventors: Amala KOMPELLA, Hyderabad (IN); Kameswara Rao VAGICHERLA, Hyderabad (IN); Tirumala Reddy LANKI REDDY, Hyderabad (IN); Narmada PESARU, Hyderabad (IN); Pulla Reddy MUDDASANI, Hyderabad (IN); Venkaiah Chowdary NANNAPANENI, Hyderabad (IN)

(21) Appl. No.: 18/556,389

(22) PCT Filed: Apr. 15, 2022
(86) PCT No.: PCT/IN2022/050363

§ 371 Date: Oct. 20, 2023

(30) Foreign Application Priority Data
Apr. 21, 2021 (IN) 202141018380

(51) Int. Cl. C07D 495/04

(52) U.S. Cl. CPC C07D 495/04

(57) ABSTRACT

The present invention describes an improved second generation process for the synthesis of NRC-1111 (1,5-[2-[[4-(methylsulfonyl)-1-piperazinyl]methyl]-7-(4-morpholinyl)thieno[2,3-c] pyridine-5-yl]-2-pyrimidinamine) dimethane sulfonate and NRC-1109 (II, 5-[3-methyl-2-[(4-methylsulfonylpiperazin-1-yl)methyl]-7-morpholino-thieno[2,3-c]pyridin-5-yl]pyrimidin-2-amine) dimethane sulfonate. This process is cost effective, high yielding and industrially feasible process for the synthesis of compounds of formulae I and II with high purity. Formula (I) Formula (I): R═H for NRC-1111 and Formula (II): R═CH3 for NRC-1109 NRC-1111 and NRC-1109 are potential anti-cancer agents.

### FIELD OF THE INVENTION

The present invention describes an improved second generation process for the synthesis of NRC-1111 (I, 5-[2-[[4-(methylsulfonyl)-1-piperazinyl]methyl]-7-(4-morpholinyl)thieno[2,3-c] pyridine-5-yl]-2-pyrimidinamine) dimethane sulfonate and NRC-1109 (II, 5-[3-methyl-2-[(4-methylsulfonylpiperazin-1-yl)methyl]-7-morpholino-thieno[2,3-c]pyridin-5-yl]pyrimidin-2-amine) dimethane sulfonate. This process is cost effective, high yielding and industrially feasible process for the synthesis of compounds of formulae I and II with high purity.

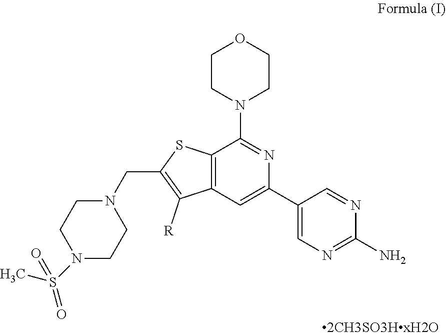

Formula (I): R═H for NRC-1111 and
Formula (II): R═CH₃ for NRC-1109;
NRC-1111 and NRC-1109 are potential anti-cancer agents.

### BACKGROUND OF THE INVENTION:

The present invention relates to an improved process for the preparation of NRC-1111 (Formula-I; R═H; 5-[2-[[4-(methylsulfonyl)-1-piperazinyl]methyl]-7-(4-morpholinyl)thieno[2,3-c]pyridine-5-yl]-2-pyrimidinamine) dimesylate hydrate and NRC-1109 (Formula-II; R═CH₃; 5-[3-methyl-2-[(4-methylsulfonylpiperazin-1-yl) methyl]-7-morpholino-thieno[2,3-c]pyridin-5-yl]pyrimidin-2-amine) dimesylate hydrate are potential anti-cancer agents.

Processes for the preparation of NRC-1111 and NRC-1109 are described in U.S. 2017/0320891 which was published on 94 Nov. 2017.
The process has been illustrated in scheme-I shown below:

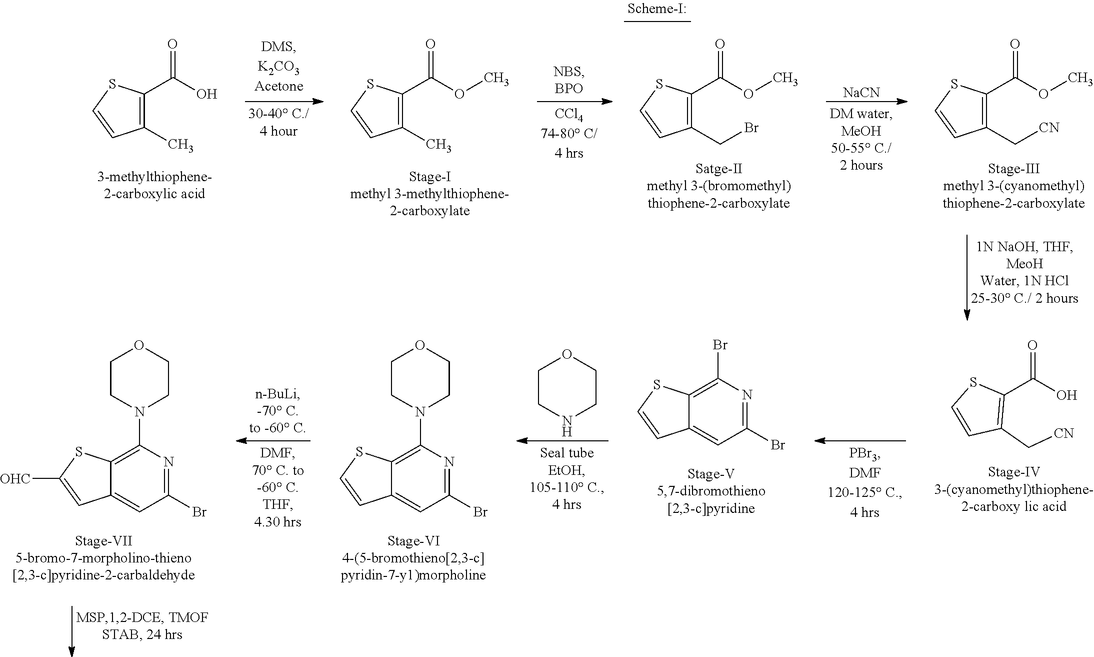

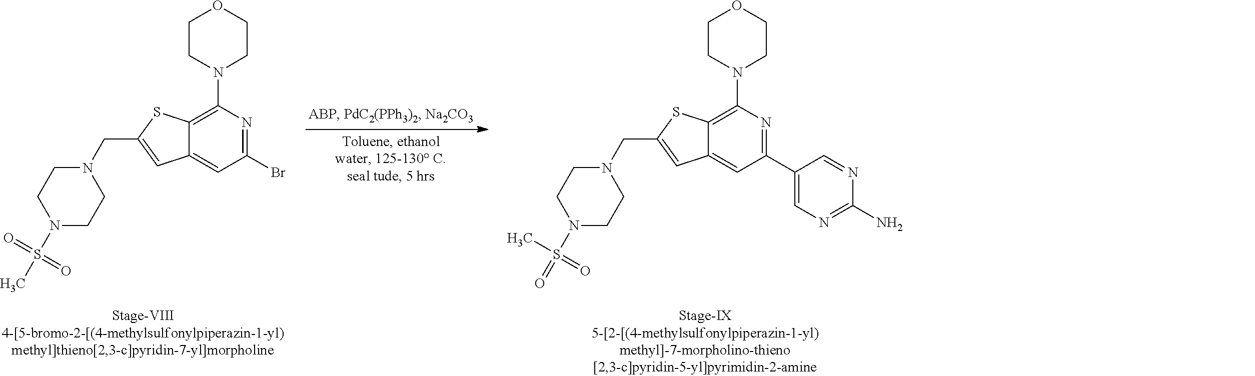 [SMILES: CS(=O)(=O)N1CCN(Cc2cc3cc(Br)nc(N4CCOCC4)c3s2)CC1 ; CS(=O)(=O)N1CCN(Cc2cc3cc(-c4cnc(N)nc4)nc(N4CCOCC4)c3s2)CC1]

The method illustrated in the scheme-I comprises (i) the esterification of 3-methylthiophene-2-carboxylic acid with dimethyl sulphate yielding methyl 3-methylthiophene-2-carboxylate (Stage-I), which is reacted with N-bromo succinimide in Carbon tetra chloride solvent affords 3-(Bromomethyl)-2-thiophenecarboxylic acid methyl ester(Stage-II). The cyanation of the Stage-II compound with Sodium cyanide in aqueous methanol gives rise to 3-(Cyanomethyl)-2-thiophenecarboxylic acid methyl ester (Stage-III) which on hydrolysis with 1N sodium hydroxide in THF and methanol medium yields 3-(Cyanomethyl)-2-thiophenecarboxylic acid (Stage-IV) on acidification. The Stage-IV compound on brominated cyclization with Phosphorous tribromide in DMF medium at 120-125° C. yields 5,7-dibromothieno[2,3-c]pyridine (Stage-V) which on condensation with Morpholine in seal tube at 105-110° C. gives 5-Bromo-7-(4-morpholinyl)-thieno[2,3-c]pyridine (Stage-VI). This stage-VI compound on lithiation with n-BuLi followed by formylation with Dimethyl formamide (DMF) gives 5-Bromo-7-(4-morpholinyl)-thieno[2,3-c]pyridine-2-carboxaldehyde (Stage-VII). This stage-VII compound on reductive amination using sodium triacetoxyborohydride (STAB) with 1-methyl sulfonyl-piperazine gives 5-Bromo-2-[[4-(methylsulfonyl)-1-piperazinyl] methyl]-7-(4-morpholinyl)-thieno[2,3-c]pyridine (Stage-VIII) which undergoes Suzuki coupling with 2-aminopyrimidine-5-boronic acid pinacol ester using bis (triphenylphosphine) Palladium (II) dichloride gives 5-[2-[[4-(methylsulfonyl)-1-piperazinyl] methyl]-7-(4-morpholinyl)thieno[2,3-c] pyridine-5-yl]-2-pyrimidinamine (NRC-1111 base).

This is first generation process and developed on small scale with overall yield of 8%. The second generation process is developed with the following objectives:
1. To develop pharmaceutically acceptable salt of NRC-1111 and NRC-1109 and identifying stable polymorph of it.
2. To develop simplified process with less number of operations which is suitable for scale up.
3. To develop cost effective and high yielding process.
4. To develop a safe process from the point of environmental protection.
5. To develop a process that should give the final product of high purity (more than 99.5%), which does not need further expensive purification
6. To develop industrially viable and robust process from the following stage-V Intermediate.

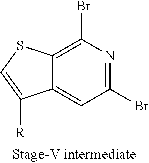

NRC-1111 process starts with above intermediate when R═H as depicted below:

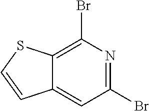 [SMILES: Brc1cc2ccsc2c(Br)n1]

5,7-dibromothieno[2,3-c]pyridine

NRC-1109 process starts with above intermediate when R═CH₃ as depicted below:

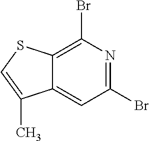

5,7-dibromo-3-methyl-thieno[2,3-c]pyridine

Thus the second generation process fulfilling the above objectives is depicted in Scheme-2:

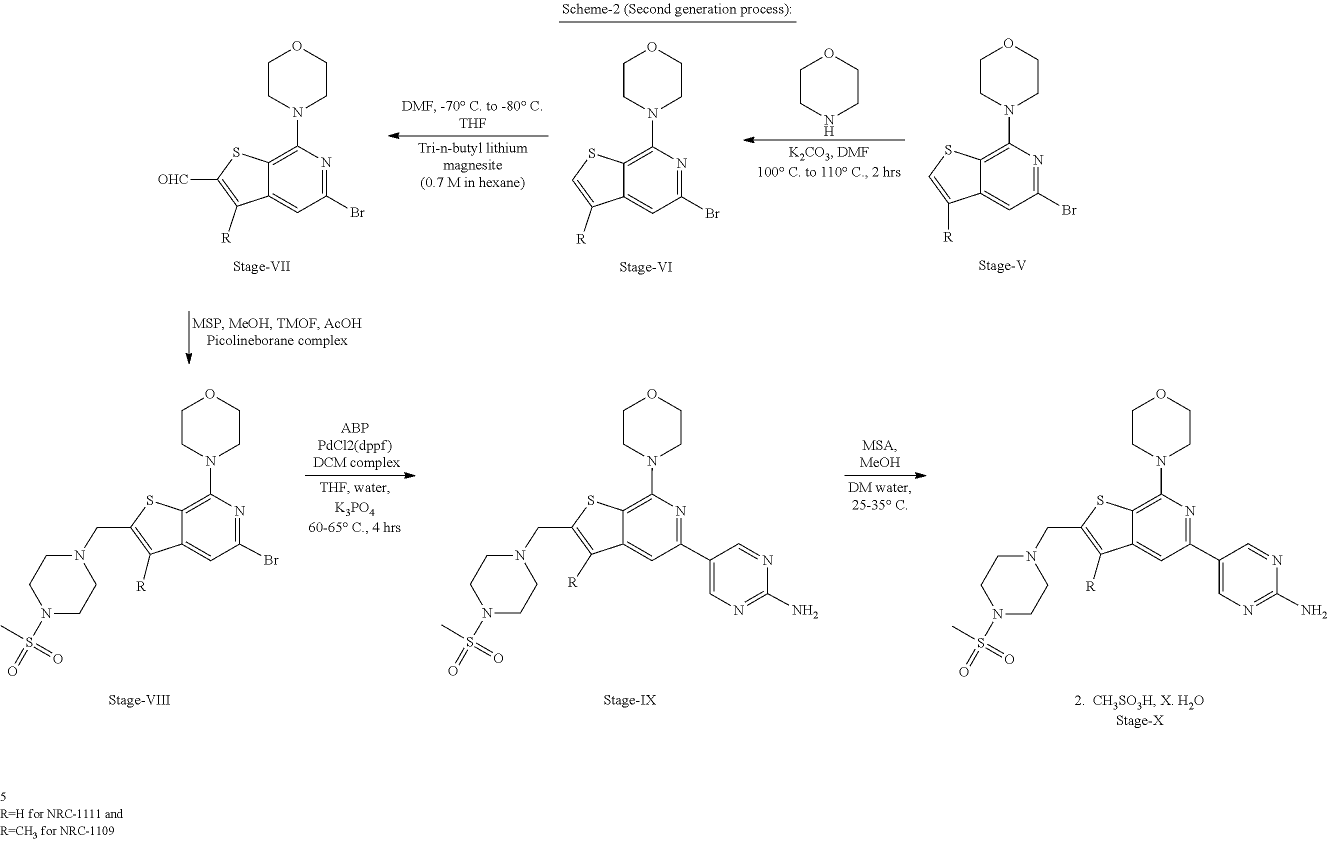

### SUMMARY OF INVENTION

Taking into consideration the above mentioned requirements, the aim of the present invention is to develop an improved process which is environmentally protective, safe, industrially applicable, devoid of the deficiencies of first generation process and makes possible the synthesis of highly pure (99.8%) desired compound of formula-I and in high yields (about 22% overall yield).

Therefore the main objective of the present invention is to develop an improved process for the preparation of NRC-1111 dimesylate hydrate which is 5-[2-[[4-(methylsulfonyl)-1-piperazinyl]methyl]-7-(4-morpholinyl)thieno[2,3-c] pyridine-5-yl]-2-pyrimidinamine dimethane sulfonate hydrate having the formula-I would be to select the scheme-II as the working route and set all the five stages of the reaction conditions, work-ups and purifications.

Still another objective of the present invention is to develop an improved process 5-Bromo-7-(4-morpholinyl)-thieno[2,3-c]pyridine (Stage-VI) by avoiding sealed tube and excess moles of Morpholine.

Another objective of the present invention is to develop an improved process for the preparation of 5-Bromo-7-(4-morpholinyl)-thieno[2,3-c]pyridine-2- carboxaldehyde (Stage-VII) using Tri n-butyllithium magnesite (0.7M in hexane) reagent for lithiation to avoid scale up problems and to get high purity product consistently.

Another objective of the present invention is to develop an improved process for the preparation of 5-Bromo-2-[[4-(methylsulfonyl)-1-piperazinyl] methyl]-7-(4-morpholinyl)-thieno[2,3-c]pyridine (Stage-VIII) by substituting STAB with 2-picoline borane complex and modifying all the process parameters.

Yet another objective of the present invention is to develop an improved process for the preparation of 5-[2-[[4-(methylsulfonyl)-1-piperazinyl]methyl]-7-(4-morpholinyl)thieno[2,3-c]pyridine-5-yl]-2-pyrimidinamine(Stage-IX; NRC-1111 base) by substituting bis(triphenylphosphine) palladium (II) dichloride with [1,1′-Bis(diphenylphosphino)ferrocene]dichloropalladium (II), complex with dichloromethane to get high yielding and pure product in multi kilogram scale.

Still another objective of the present invention is to select dimesylate as acid addition salt of NRC-1111 and NRC-1109 and to make corresponding hydrates. Thus the polymorphic nature of both NRC-1111 dimesylate hydrate and NRC-1109 dimesylate hydrate are established on kilogram scale.

Another objective of the present invention is to provide crystal form of 5-[2-[(4-methylsulfonylpiperazin-1-yl)methyl]-7-morpholino-thieno [2,3-c] pyridin-5-yl] pyrimidin-2-amine) (NRC-1111 base) is characterized by:
i) Its powder X-ray diffractogram having peaks at about 9.31, 11.06, 18.64 and 20.05±0.2 degrees 2-theta.
ii) powdered X-ray diffraction pattern as shown in figure-1.

Another objective of the present invention is to provide crystal form of 5-[3-methyl-2-[(4-methylsulfonylpiperazin-1-yl)methyl]-7-morpholino-thieno [2,3-c] pyridin-5-yl]pyrimidin-2-amine) (NRC-1109 base) is characterized by:
i) Its powder X-ray diffractogram having peaks at about 8.76, 9.45, 16.87, 18.39 and 19.50±0.2 degrees 2-theta.
ii) powdered X-ray diffraction pattern as shown in figure-3.

Another objective of the present invention is to provide crystal form of 5-[2-[(4-methylsulfonylpiperazin-1-yl)methyl]-7-morpholino-thieno [2,3-c]pyridin-5-y] pyrimidin-2-amine) dimethane sulfonate hydrate (NRC-1111) is characterized by:
i) Its powder X-ray diffractogram having peaks at about 6.19, 15.11, 18.40, 18.65, 21.60, 22.56 and 25.13±0.2 degrees 2-theta.
ii) powdered X-ray diffraction pattern as shown in figure-5.

Another objective of the present invention is to provide crystal form of 5-[3-methyl-2-[(4-methylsulfonylpiperazin-1-yl)methyl]-7-morpholino-thieno [2,3-c]pyridin-5-yl]pyrimidin-2-amine) dimethane sulfonate hydrate (NRC-1109) is characterized by:
i) Its powder X-ray diffractogram having peaks at about 7.67, 10.69, 18.39, 19.58, 21.93 and 25.83±0.2 degrees 2-theta.
ii) powdered X-ray diffraction pattern as shown in figure-7.

Another objective of the present invention is to provide a process for the preparation of crystal form of 5-[2-[(4-methylsulfonylpiperazin-1-yl)methyl]-7-morpholino-thieno [2,3-c]pyridin-5-yl]pyrimidin-2-amine) dimethane sulfonate hydrate (NRC-1111), comprising the steps of:
a) adding DM water to 5-[2-[(4-methylsulfonylpiperazin-1-yl)methyl]-7-morpholino-thieno [2,3-c]pyridin-5-yl]pyrimidin-2-amine (NRC-1111 base),
b) heating the reaction mixture to 90-100° C.,
c) cooling the reaction mixture and adding methanol,
d) adding methanesulfonic acid diluted in methanol to the reaction mixture,
e) stirring and filtering the mass to get the crystal form of NRC-1111 dimethane sulfonate hydrate.

In Stage-VI for the preparation of 5-Bromo-7-(4-morpholinyl)-thieno[2,3-c] pyridine the number of moles of Morpholine to 5,7-dibromothieno[2,3-c]pyridine (Stage-V) the methacrylic acid may be in the range of 2.0-3.0 preferably in the range of 2.25-2.50 moles. The number of moles of Potassium Carbonate to Stage-V may be in the range of 2.0-4.0 preferably in the range of 2.0-3.0. Preferred solvent for the reaction is Dimethyl formamide (DMF).

In Stage-VII for the preparation of 5-Bromo-7-(4-morpholinyl)-thieno[2,3-c]pyridine-2-carboxaldehyde the moles of 0.7M tri-n butyllithummagnesate in hexane may be in the range of 0.9-1.3 preferably in the range of 1.1-1.2 moles. The moles of dimethyl formamide may be in the range of 1.8-2.2 preferably 1.9-2.1 moles. Preferred solvent for the reaction is Tetrahydrofuran (THF). Alternatively stage-VI can be prepared using isopropylmagnesium bromide (1.5M in THF) n-butyl lithium (1.6M in hexane) reagents.

In Stage-VIII for the preparation of 5-Bromo-2-[[4-(methylsulfonyl)-1-piperazinyl] methyl]-7-(4-morpholinyl)-thieno[2,3-c]pyridine the number of moles of 1-methane sulfonylpiperazine may be in the range of 1.4-1.8 preferably in the range of 1.2-1.6. The number of moles of trimethyl orthoformate may be in the range of 14-18 moles preferably in the range of 15-17. The number of moles of 2-Picoline borane complex may be in the range of 2.5-3.5 moles preferably in the range of 2.8-3.2

In Stage-IX for the preparation of 5-[2-[[4-(methylsulfonyl)-1-piperazinyl] methyl]-7-(4-morpholinyl)thieno[2,3-c] pyridine-5-yl]-2-pyrimidinamine (NRC-1111 base) the number of moles of [1,1′-Bis(diphenylphosphino)ferrocene] dichloropalladium (II), complex with dichloromethane may be in the range of 0.01 to 0.05 moles preferably 0.05 moles. The base for the reaction may be selected from Potassium phosphate, Potassium acetate or potassium carbonate Preferably Potassium phosphate.

In Stage-X for the preparation of 5-[2-[[4-(methylsulfonyl)-1-piperazinyl] methyl]-7-(4-morpholinyl)thieno [2,3-c] pyridine-5-yl]-2-pyrimidinamine dimesylate hydrate corresponding base is refluxed with water to get hydrate followed by treatment with methanesulfonic acid to get dimesylate hydrate.

### BRIEF DESCRIPTION OF THE DRAWINGS

FIG. 1: Illustrates the characteristic PXRD pattern of NRC-1111 base crystal form.

FIG. 2: Illustrates the DSC thermogram of NRC-1111 base crystal form.

FIG. 3: Illustrates the characteristic PXRD pattern of NRC-1109 base crystal form.

FIG. 4: Illustrates the DSC thermogram of NRC-1109 base crystal form.

FIG. 5: Illustrates the characteristic PXRD pattern of NRC-1111 dimesylate hydrate crystal form.

FIG. 6: Illustrates the DSC thermogram of NRC-1111 dimesylate hydrate crystal form.

FIG. 7: Illustrates the characteristic PXRD pattern of NRC-1109 dimesylate hydrate crystal form.

FIG. 8: Illustrates the DSC thermogram of NRC-1109 dimesylate hydrate crystal form.

### PXRD Method of Analysis

PXRD analysis of the crystalline forms of NRC-1111 & NRC-1109 and its salts were carried out using Panlytical Expert Pro DY3248 X-ray powder diffractometer using Cu-Ka radiation of 10 wavelength 1.5406 A° and at continuous scan speed of 0.03°/min.

The details of the inventions are given in the Example provided below which are provided for illustration only and therefore these examples should not be construed to limit the scope of the invention.

### EXAMPLES

### Example-1: Process for the Preparation of Compound of Stage-VI [Where R═H]

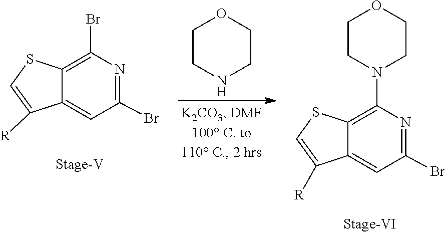

Charge dimethylformamide 425 ml and 5,7-dibromothieno[2,3-c]pyridine 85.0 (0.2901 mol) at 25-35° C.and sir for 5-10 min. Charge potassium carbonate 100.22 g (0.7252 mol) followed by morpholine 63.16 g (0.7252 mol) at 25-35° C. Raise the mass temperature to 100-110° C. for 2 hrs. Cool the reaction mass to RT and filter the reaction mass. Wash the wet cake with methanol and charge filtrate into DM water 2975 ml at 20-25° C. and stir for 30min. Filter the solid and wash with methanol to give light brown solid 80.55 g (92.78%) with 98.11% HPLC purity.

### Example-2: Process for the Preparation of Compound of Stage-VI [R═CH₃]

Charge N,N-Dimethylformamide (625 ml) and 5,7-dibromo 3-methylthieno[ 2, 3-c] pyridine 125 g (0.4071 mol) at 25-35° C. and sir for 5-10 min. Charge potassium carbonate 168.81 g (1.221 mol) followed by morpholine 106.42 g (1.221 mol) at 25-35° C. Raise the mass temperature to 110-115° C. for 2 hrs. Cool the reaction mass to RT and filter the reaction mass. Wash the wet cake with methanol. Charger filtrate into DM water 2975 ml at 20-25° C. and stir for 90 min. Filter the solid and wash with methanol to give light brown solid 120.01 g (94.11%) with 98.68% HPLC purity.

### Example-3: Process for the Preparation of Compound of Stage-VII [where R═H]

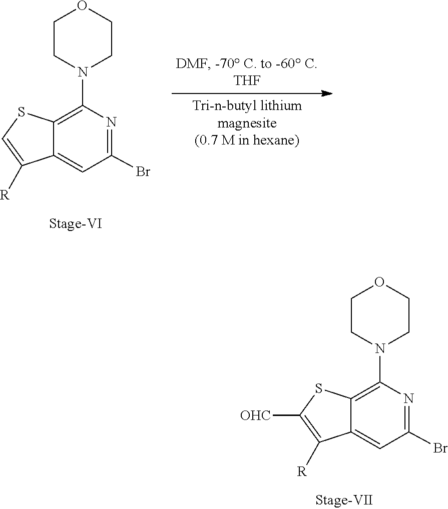

Charge dry tetrahydrofuran 440 ml and 4-(5-bromothieno[2,3-c]pyridin-7-yl) morpholine 44.0 g (0.147 mol) at 25-35° C. Cool the mass temp to −60° to −70° C. and charge Tri n-butyllithium magnesate (0.7M in hexane) 234.34 ml (0.1617 mol). Stir the reaction mass for 90 min, charge dry dimethylformamide 21.48 g (0.294 mol) and stir for 90 min. Raise the mass temp to 0-5° C. and stir for 45 min. Charge 2N HCl to the reaction mass at 0-5° C. and stir for 15 min. Extract with ethyl acetate and wash with 10% sodium chloride solution. Distill off organic layer and charge Isopropyl ether and Tetrahydrofuran mixture to the residue. Raise mass temperature to 55-60° C., bring mass to RT and charge hexane to the reaction mass .Filter the solid and wash with hexane to give yellow solid 25.42 g (52.48%) with 99% HPLC purity.

### Example-4: Alternate Process for the Preparation of Compound of Stage-VII [Where R═H]

Charge dry tetrahydrofuran 200 ml and 4-(5-bromothieno[2,3-c]pyridin-7-yl) morpholine 20 g (0.0668 mol) at 25-35° C. Cool the mass temp to −60° to −70° C. and charge isopropylmagnesium bromide (1.5M in THF) 24.96 ml (0.0367 mol) followed by n-butyl lithium (1.6M in hexane) 45.95 ml (0.0735 mol). Stir the reaction mass for 90 min, charge dry dimethylformamide 9.76 g (0.1336 mol) and stir for 90 min. Raise the mass temp to 0-10° C. and stir for 45 min. Charge 2N HCl to the reaction mass at 0-10° C. and stir for 15 min. Extract with ethyl acetate and wash with 10% sodium chloride solution. Distill off organic layer and charge Isopropyl ether and Tetrahydrofuran mixture to the residue. Raise mass temperature to 55-60° C., bring mass to RT and charge hexane to the reaction mass. Filter the solid and wash with hexane to give yellow solid 16.02 g (73.25%) with 98.8% HPLC purity.

### Example-5: Process for the Preparation of Compound of Stage-VII [R═CH₃]

Charge dry tetrahydrofuran 2200 ml and 4-(5-bromo-3-methylthieno-[2,3-c] pyridine -7-yl)-morpholine 110 g (0.3512 mol) at 25-35° C. Cool the mass temp to −65° to −71° C. and charge slowly Tri n-butyllithium magnesate (0.7M in hexane) 451.52 ml (0.3161 mol). Stir the reaction mass for 30 min, charge slowly dry N, N-Dimethylformamide 54.62 ml (0.7024 mol) and stir for 120 min. Raise the mass temp to −15 to −25° C. and quench with 2N HCl 2200 ml to the reaction mass at −15 to −25° C. and stir for 15 min. Extract with dichloromethane and wash organic layer with DM water. Distil off organic layer, charge methanol to the residue and filter the yellow solid. Yield: 75.6 g (63%) with 97.8% HPLC purity

### Example-6: Alternate Process for the Preparation of Compound of Stage-VII [R═CH₃]

Charge dry tetrahydrofuran 500 ml and 4-(5-bromothieno[2,3-c]pyridin-7-yl) morpholine 50 g (0.159 mol) at 25-35° C. Cool the mass temp to −60° to −70° C. and charge isopropylmagnesium bromide (1.5M in THF) 70.69 g (0.087 mol) followed by n-butyl lithium (1.6M in hexane) 52.5 g (0.175 mol). Stir the reaction mass for 90 min, charge dry dimethylformamide 23.3 g (0.319 mol) and stir for 90 min. Raise the mass temp to 0-10° C. and stir for 45 min. Charge 2N HCl to the reaction mass at 0-10° C. and stir for 15 min. Extract with ethyl acetate and wash with 10% sodium chloride solution. Distill off organic layer and charge Isopropyl ether and Tetrahydrofuran mixture to the residue. Raise mass temperature to 55-60° C., bring mass to RT and charge hexane to the reaction mass. Filter the solid and wash with hexane to give yellow solid 35.4 g (65%) with 98.10% HPLC purity.

### Example-7: Process for the Preparation of Compound of Stage-VIII [R═H]

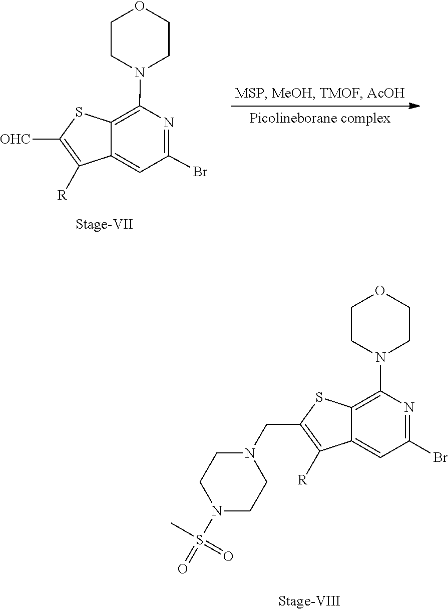

Charge methanol 650 ml and 5-bromo-7-morpholino-thieno[2,3-c]pyridine-2-carbaldehyde 65 g (0.1986 mol) at 25-35° C. Charge 1-methane sulfonylpiperazine 52.19 g (0.3178) followed by trimethyl orthoformate 316.07 g (2.979 mol) and acetic acid 11.90 g (0.1986 mol) and stir for 4 hrs at 55-60° C. Dissolve 2-picoline borane complex 63.72 g (0.5958) in THF 130 ml and charge to the reaction mass at 55-60° C. and stir for 3 hrs. Cool the mass to RT and filter the reaction mass and wash with methanol. Charge 975.0 ml dichloromethane and crude solid followed by conc HCl 39.65 ml at 25-30° C. Raise the temperature to reflux and maintain for 30 min, bring to RT and filter. Charge wet compound into DM water and neutralize to PH 9-10 with aq ammonia at 25-30° C. Filter the solid and wash with DM water dry at 55-60° C. to give yellow color compound 77.24 g (81.8%) with 99.4% HPLC purity.

### Example-8: Process for the Preparation of Compound of Stage-VIII [R═CH₃]

Charge methanol 1200 ml and 5-bromo-3-methyl-7-morpholino-thieno[2,3-c] pyridine 60 g (0.1758 mol) at 25-35° C. Charge 1-methane sulfonylpiperazine 46.20 g (0.281 mol) followed by trimethyl orthoformate 279.87 g (2.637 mol) and acetic acid 10.55 g (0.1758 mol) and stir for 4 hrs at 60-65° C. Dissolve 2-picoline borane complex 56.43 g (0.528) in THF 120 ml and charge slowly to the reaction mass at 60-65° C. and stir for 3 hrs. Cool the mass to RT and filter the reaction mass and wash with methanol. Charge 975 ml dichloromethane and crude solid followed by conc HCl 39.65 ml at 25-30° C. Raise the temperature to reflux and maintain for 30 min, bring to RT and filter. Charge wet compound into DM water and neutralize to P^{H} 9- 10 with aq ammonia at 25-30° C. Filter the solid and wash with DM water and dry at 55-60° C. to give yellow color compound 63.84 g (74.2%) with 98.5% HPLC purity.

### Example-9: Process for the Preparation of Compound of Stage-IX [where R═H]

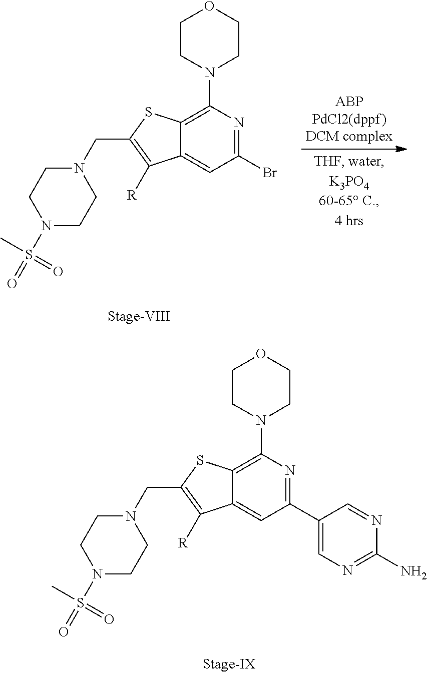

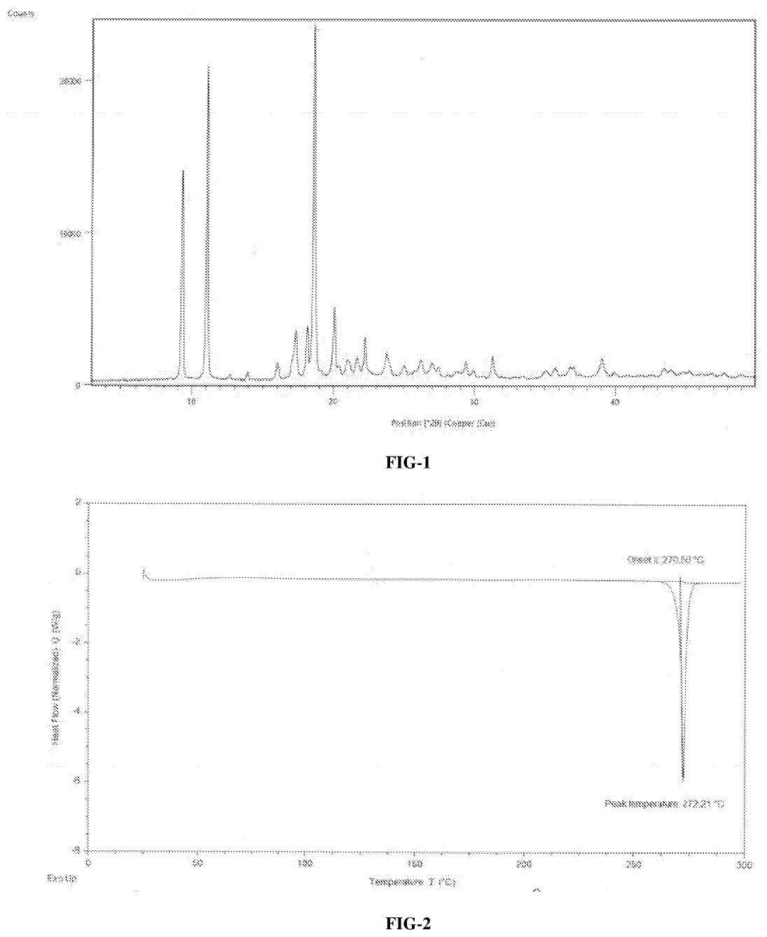

Charge Potassium phosphate 64.31 g (0.303 mol), DM water 86.4 ml and tetrahydrofuran 720 ml and de-gasify under nitrogen atm for 60 min at 25-30° C. Charge 4-[5-bromo-2[(4-methylsulfonylpiperazin-1-yl)methyl]thieno[2,3-c] pyridin-7-yl] morpholine 72 g (0.1515) and 2-aminopyrimidine-5-boronic acid pinacol ester 40.18 g (0.1818 mol) de-gasify under nitrogen atmosphere for 45 min at 25-30° C. Charge [1,1′-Bis(diphenylphosphino)ferrocene]dichloropalladium (II), complex with dichloromethane 6.186 (0.00757 mol) to the reaction mass and de-gasify under nitrogen atmosphere for 10 min at 25-30° C. Raise the mass temperature to 60-65° C. for 5 hrs under nitrogen atm. The reaction mixture is cooled to RT and charged DM water 720 ml and filtered the solid wash with DM water. The crude solid is purified by acid-base purification method to give 53.09 g (71.5%) with 99.8% HPLC purity. XRD and DSC are depicted by FIGS. 1 and 2.

### Example-10: Process for the Preparation of Compound of Stage-IX [R═CH₃]

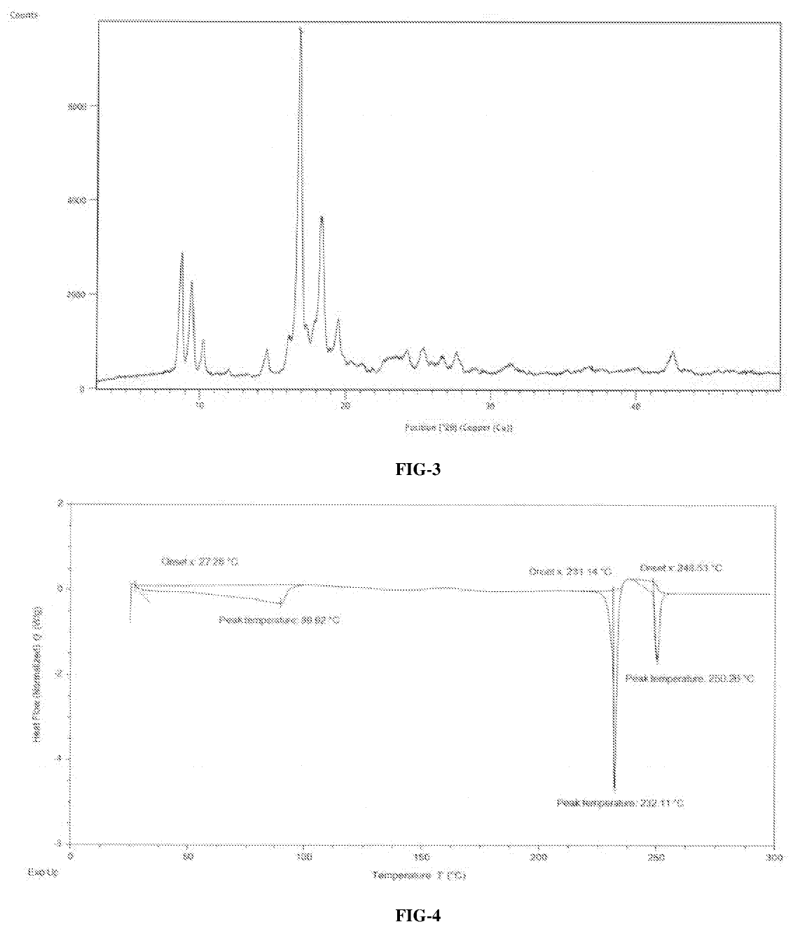

Charge Potassium phosphate 47.71 g (0.2247 mol), DM water 66 ml and tetrahydrofuran 550 ml and de-gasify under nitrogen atmosphere for 60 min at 25-30° C. Charge 4-[5-bromo 3-methyl-2-[(4-methylsulfonylpiperazin-1-yl)methyl] thieno[2,3-c]pyridin-7-yl]morpholine 55 g (0.1123) and 2-aminopyrimidine-5-boronic acid pinacol ester 29.81 g (0.1348 mol) de-gasify under nitrogen atm for 45 min at 25-30° C. Charge [1, 1′-Bis (diphenylphosphino) ferrocene]dichloropalladium (II), complex with dichloromethane 4.59 g (0.00562 mol) to the reaction mass and de-gasify under nitrogen atm for 10 min at 25-30° C. Raise the mass temperature to 60-65° C. for 5 hrs under nitrogen atm. The reaction mixture is cooled to RT and filter. The crude solid is purified by acid-base purification method to give 43.1 g (76.2%) with 99.81% HPLC purity. XRD and DSC are depicted by FIGS. 3 and 4.

### Example-11: Process for the Preparation of Compound of Stage-X [Where R═H]

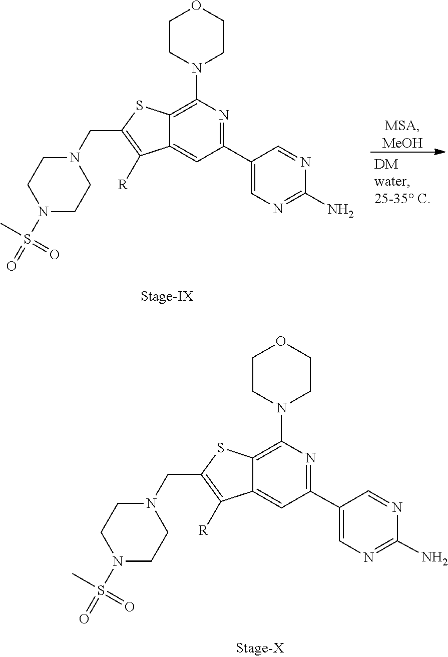

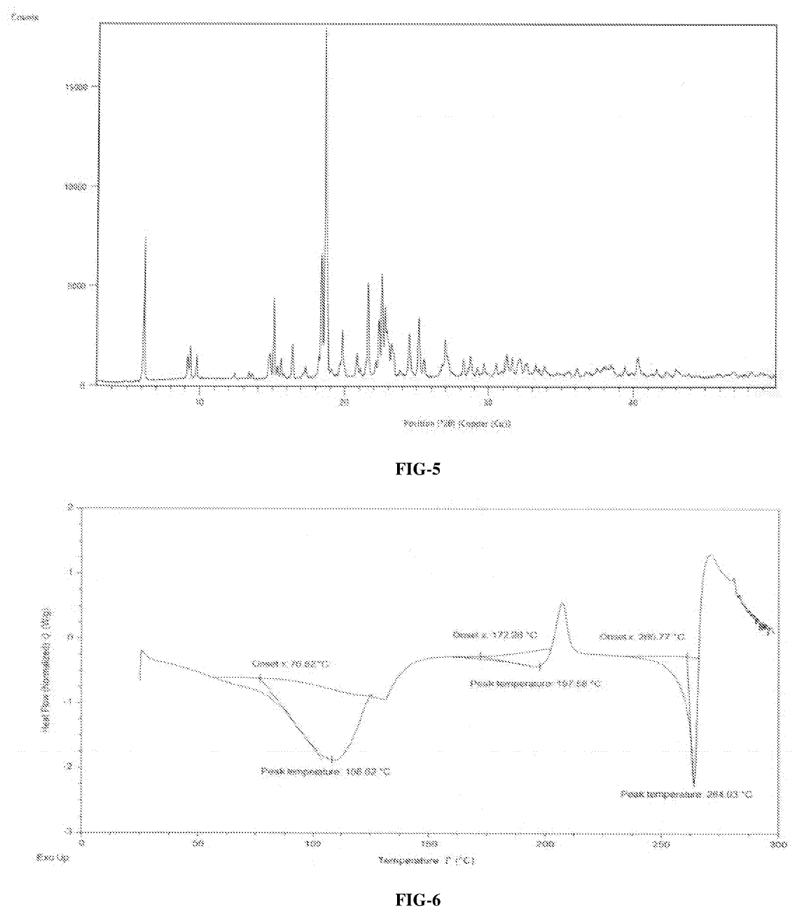

Charge DM water 350 ml and 5-[2-[(4-methylsulfonylpiperazin-1-yl)methyl]-7-morpholino-thieno [2,3-c]pyridin-5-yl]pyrimidin-2-amine 140 g(0.285 mol) at 25-30° C., stir for 5-10 min. Raise the reaction mass temperature to 90-100° C. for 1 hr. Cool the reaction mass to RT and stir for 2hr. Charge methanol 2800 ml at 25-30° C. and stir for 5-10 min. Charge methanesulfonic acid 109.92 (1.143 mol) diluted in methanol 420 ml to the reaction mass at 25-30° C. Stir the reaction mass for 2 hrs and filter the reaction mass and wash with methanol 140 ml to give 5-[2-[(4-methylsulfonylpiperazin-1-yl)methyl]-7-morpholino-thieno[2,3-c]pyridin-5-y] pyrimidin-2-amine dimesylate hydrate 208.87 g (1.5% w/w) and with 99.9% HPLC purity, assay by HPLC is 101.4% w/w. Water content by Karl-Fischer: 10%; XRD and DSC are depicted by FIGS. 5 and 6.

### Example-12: Process for the Preparation of Compound of Stage-X [R═CH₃]

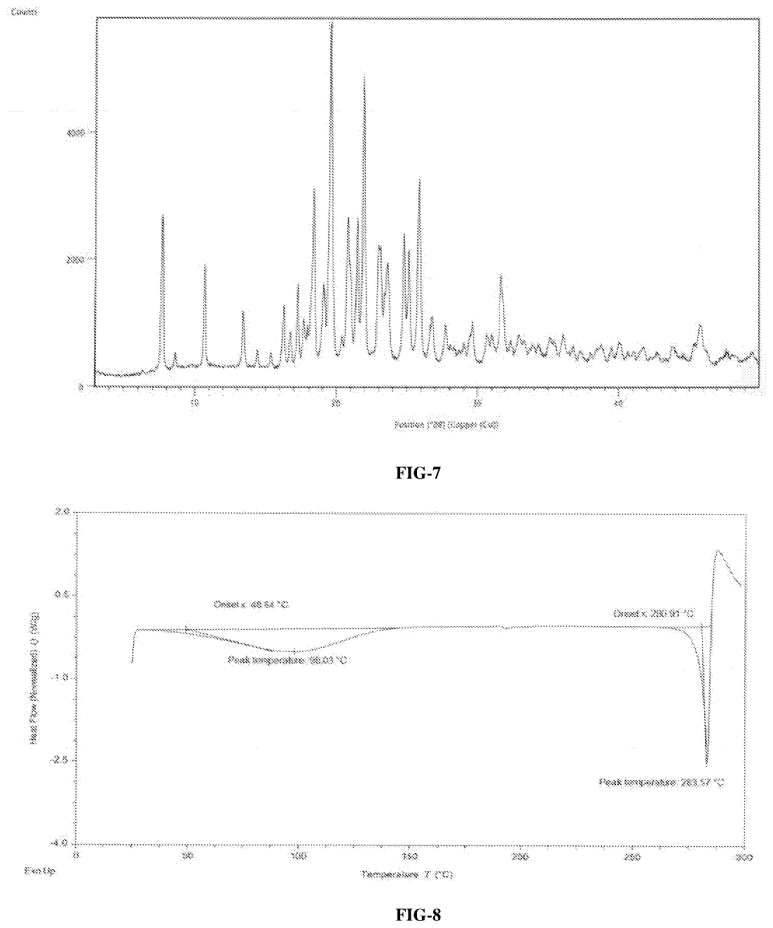

Charge methanol 750 ml and 5-[3-methyl-2-[(4-methylsulfonylpiperazin-1-yl) methyl]-7 morpholino-thieno[2,3-c]pyridin-5-yl]pyrimidin-2-amine 30 g (0.0595 mol) and DM water 2.41 g (0.119 mol) at 25-30° C., stir for 5-10 min. Charge methanesulfonic acid 14.31 g (0.1489 mol) diluted in methanol 150 ml to the reaction mass at 25-30° C. Raise the reaction mass temperature to 60-65° C. for 1 hr. Filter the reaction mass at 45° C. and wash with methanol 300 ml to give 5-[3-methyl-2-[(4-methylsulfonylpiperazin-1-yl)methyl]-7 morpholino-thieno[2,3-c]pyridin-5-yl] pyrimidin-2-amine dimesylate hydrate 37.93 g (1.26% w/w) and with 99.8% HPLC purity; Assay by HPLC is 100.0% w/w. Water content by Karl-Fischer: 5%; XRD and DSC are depicted by FIGS. 7 and 8.

### Advantages of the Present Invention:

1. Stabilization in multi kilogram scale level.
2. High yielding (about ˜20% overall yield) process.
3. NRC-1111 dimesylate hydrate and NRC-1109 dimesylate hydrate of high purity (99.8%) is obtained.
4. Simple and industrially applicable process.

1. An improved process for the preparation of 5-[3-substituted-2-[[4-(methylsulfonyl)-1-piperazinyl]methyl]-7-(4-morpholinyl)thieno[2,3-c]pyridine-5-yl]-2-pyrimidinamine)dimethane sulfonate compound of Formula-(I), 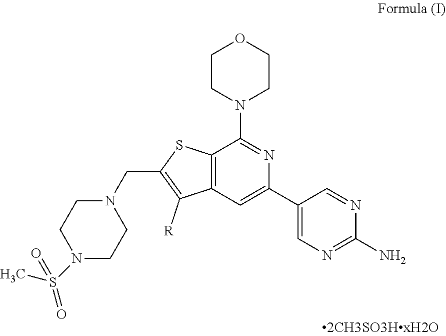
Formula (I): R═H for NRC-1111 and
Formula (II): R═CH₃ for NRC-1109;
comprising the following steps of:
a) Reacting the compound offormula-(V) 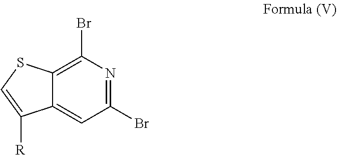
Formula (V): R═H for NRC-1111 and
:R═CH₃ for NRC-1109;
with morpholine in presence of a suitable base and a solvent provides compound of formula-(VI), 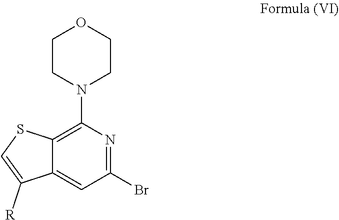
Formula (VI): R═H for NRC-1111 and
:R═CH₃ for NRC-1109;
b) treating the compound of formula-(VI) with a suitable base in a solvent followed by formylation with dimethylformamide provides compound of formula-(VII),
(or)
treating the compound of formula-(VI) with a Grignard reagent and a suitable base in a solvent followed by formylation with dimethylformamide provides compound of formula-(VII), 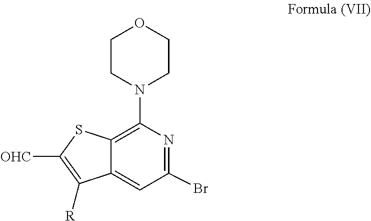
Formula (VII): R═H for NRC-1111 and
:R═CH₃ for NRC-1109;
c) reacting the compound of formula-(VII) with 1-methyl sulfonyl-piperazine in presence of 2-picoline borane complex, trimethyl orthoformate in a solvent provides compound of formula-(VIII), 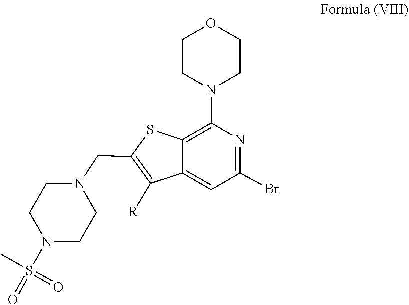
Formula (VIII): R═H for NRC-1111 and
:R═CH₃ for NRC-1109;
d) reacting the compound of formula-(VIII) with 2-aminopyrimidine-5-boronic acid pinacol ester in presence of PdC12(dppf)DCM complex, a suitable base and a solvent provides compound of formula-(IX), 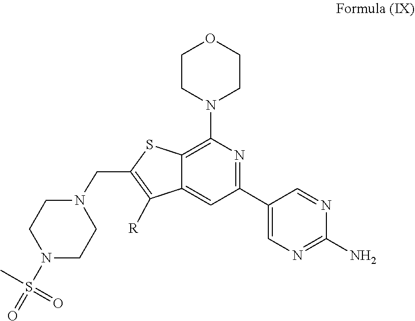
Formula (IX): R═H for NRC-1111 and
:R═CH₃ for NRC-1109;
e) treating the compound of formula-(IX) with methane sulfonic acid in a solvent provides compound of formula-(I).

2. The process as claimed in claim 1, wherein,
In step (a), the base is inorganic base. Wherein, the inorganic base is selected from alkali and alkaline metal carbonates such as sodium bicarbonate, sodium carbonate, potassium bicarbonate, potassium carbonate, cesium carbonate, or any other equivalent base.
In step (b), the base is selected from n-butyl lithium, Tri n-butyllithium magnesate; And the Grignard regent is alkylmagnesuim halide, preferably isopropylmagnesium bromide.
In step (d), the base is inorganic base selected from Potassium phosphate, Potassium acetate or potassium carbonate Preferably Potassium phosphate.
In step-a, b, c, d & e) the suitable solvent is selected from alcoholic solvents, polar-aprotic solvents, ether solvents, hydrocarbon solvents, nitrile solvents and polar solvents such as water or mixtures thereof;

3. The process as claimed in claim 1, wherein in step-a) the number of moles of Morpholine to formula-(V) is in the range of 2.0-3.0 preferably in the range of 2.25-2.50 moles. And the number of moles of Potassium Carbonate to formula-V is in the range of 2.0-4.0 preferably in the range of 2.0-3.0.

4. The process as claimed in claim 1, wherein in step-b) the number of moles of formula-(VI) to Tri-n butyllithum magnesate in hexane is in the range of 0.9-1.3 preferably in the range of 1.1-1.2 moles. And the number of moles of dimethyl formamide to formula-(VI) is in the range of 1.8-2.2 preferably 1.9-2.1 moles.

5. The process as claimed in claim 1, wherein in step-c) the number of moles of formula-(VII) to 1-methane sulfonylpiperazine is in the range of 1.4-1.8 preferably in the range of 1.2-1.6. And the number of moles of formula-(VII) to trimethyl orthoformate is in the range of 14-18 moles preferably in the range of 15-17. The number of moles of formula-(VII) to 2-Picoline borane complex is in the range of 2.5-3.5 moles preferably in the range of 2.8-3.2.

6. The process as claimed in claim 1, wherein in step-d) the number of moles of formula-(VIII) to [1,1′-Bis(diphenylphosphino)ferrocene] dichloropalladium (II), complex with dichloromethane is in the range of 0.01 to 0.05 moles, preferably 0.05 moles.

7. The process as claimed in claim 1, wherein the compound of formula-(VI) is prepared by reacting the compound of formula-(V) with morpholine in presence of a suitable inorganic base selected from alkali and alkaline metal carbonates such as sodium bicarbonate, sodium carbonate, potassium bicarbonate, potassium carbonate, cesium carbonate and a solvent is polar-aprotic solvents such as dimethylformamide.

8. The process as claimed in claim 1, wherein compound of formula-(VII) is prepared by treating the compound of formula-(VI) with Tri n-butyllithium magnesate in tetrahydrofuran followed by formylation with dimethylformamide provides compound of formula-(VII).

9. The process as claimed in claim 1, wherein compound of formula-(VII) is prepared by treating the compound of formula-(VI) with isopropylmagnesium bromide and n-butyl lithium in tetrahydrofuran followed by formylation with dimethylformamide provides compound of formula-(VII).

10. The process as claimed in claim 1, wherein compound of formula-(VIII) is prepared by reacting the compound of formula-(VII) with 1-methyl sulfonyl-piperazine in presence of 2-picoline borane complex, trimethyl orthoformate and acetic acid in methanol provides compound of formula-(VIII).

11. The process as claimed in claim 1, wherein compound of formula-(IX) is prepared by reacting the compound of formula-(VIII) with 2-aminopyrimidine-5-boronic acid pinacol ester in presence of PdC12(dppf)DCM complex, potassium phosphate in tetrahydrofuran provides compound of formula-(IX).

12. The process as claimed in claim 1, wherein compound (NRC-1111) of formula-(I) is prepared by treating the compound of formula-(IX) with methane sulfonic acid in aqueous methanol provides (NRC-1111) dimethane sulfonate compound of formula-(I).

13. The process as claimed in claim 1 for the preparation of 5-[2-[[4-(methylsulfonyl)-1-piperazinyl]methyl]-7-(4-morpholinyl)thieno[2,3-c] pyridine-5-yl]-2-pyrimidinamine) dimethane sulfonate (NRC-1111), 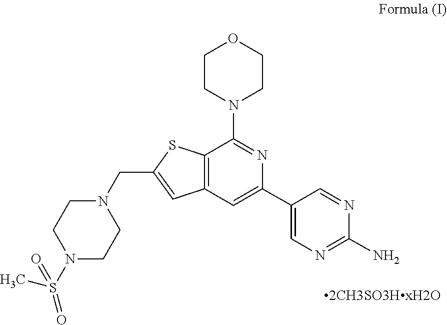 [SMILES: CS(=O)(=O)N1CCN(Cc2cc3cc(-c4cnc(N)nc4)nc(N4CCOCC4)c3s2)CC1]
comprising the following steps of:
a) Reacting the compound of formula-(V) 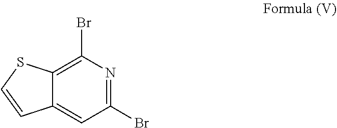 [SMILES: Brc1cc2ccsc2c(Br)n1]
with morpholine in presence of potassium carbonate base in dimethylformamide solvent provides compound of formula-(VI), 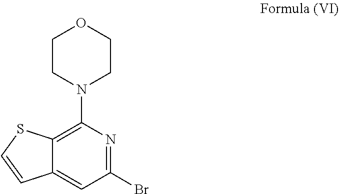 [SMILES: Brc1cc2ccsc2c(N2CCOCC2)n1]
b) treating the compound of formula-(VI) with Tri n-butyllithium magnesate reagent in tetrahydrofuran solvent followed by formylation with dimethylformamide provides compound of formula-(VII),
(or)
treating the compound of formula-(VI) with reagents isopropylmagnesium bromide and n-butyl lithium in tetrahydrofuran solvent followed by formylation with dimethylformamide provides compound of formula-(VII), 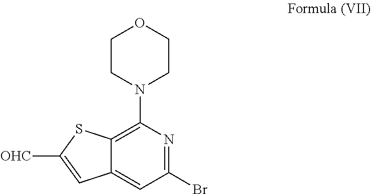 [SMILES: O=Cc1cc2cc(Br)nc(N3CCOCC3)c2s1]
c) reacting the compound of formula-(VII) with 1-methyl sulfonyl-piperazine in presence of reagents 2-picoline borane complex, trimethyl orthoformate and acetic acid in methanol solvent provides compound of formula-(VIII), 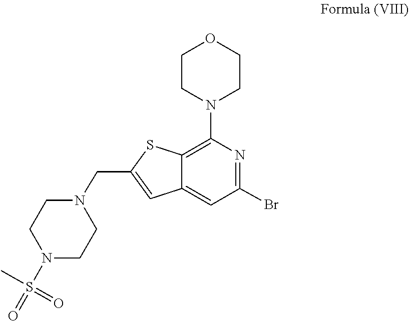 [SMILES: CS(=O)(=O)N1CCN(Cc2cc3cc(Br)nc(N4CCOCC4)c3s2)CC1]
d) reacting the compound of formula-(VIII) with 2-aminopyrimidine-5-boronic acid pinacol ester in presence of the catalyst PdC12(dppf)DCM complex and potassium phosphate base in tetrahydrofuran solvent provides compound of formula-(IX), 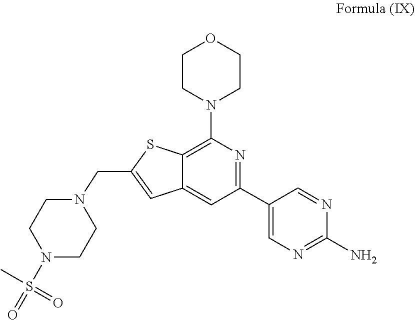 [SMILES: CS(=O)(=O)N1CCN(Cc2cc3cc(-c4cnc(N)nc4)nc(N4CCOCC4)c3s2)CC1]
e) treating the compound of formula-(IX) with methane sulfonic acid in aqueous methanol provides (NRC-1111) compound of formula-(I).

14. The process as claimed in claim 1 for the preparation of 5-[3-methyl-2-[[4-(methylsulfonyl)-1-piperazinyl]methyl]-7-(4-morpholinyl)thieno[2,3-c] pyridine-5-yl]-2-pyrimidinamine) dimethane sulfonate (NRC-1109), 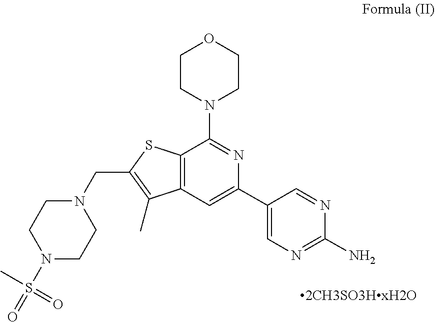 [SMILES: Cc1c(CN2CCN(S(C)(=O)=O)CC2)sc2c(N3CCOCC3)nc(-c3cnc(N)nc3)cc12]
comprising the following steps of:
a) Reacting the compound of formula-(V) 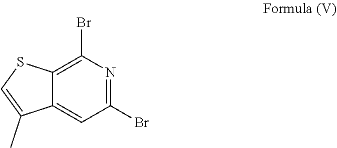
with morpholine in presence of potassium carbonate base in dimethylformamide solvent provides compound of formula-(VI), 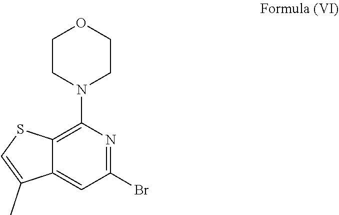 [SMILES: Cc1csc2c(N3CCOCC3)nc(Br)cc12]
b) treating the compound of formula-(VI) with Tri n-butyllithium magnesate reagent in tetrahydrofuran solvent followed by formylation with dimethylformamide provides compound of formula-(VII),
(or)
treating the compound of formula-(VI) with reagents isopropylmagnesium bromide and n- butyl lithium in tetrahydrofuran solvent followed by formylation with dimethylformamide provides compound of formula-(VII), 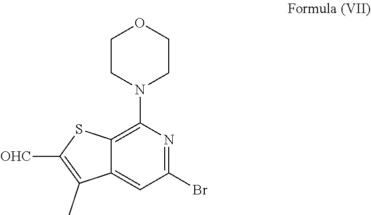 [SMILES: Cc1c(C=O)sc2c(N3CCOCC3)nc(Br)cc12]
c) reacting the compound of formula-(VII) with 1-methyl sulfonyl-piperazine in presence of reagents 2-picoline borane complex, trimethyl orthoformate and acetic acid in methanol solvent provides compound of formula-(VIII), 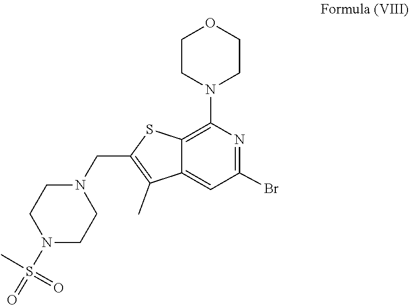 [SMILES: Cc1c(CN2CCN(S(C)(=O)=O)CC2)sc2c(N3CCOCC3)nc(Br)cc12]
d) reacting the compound of formula-(VIII) with 2-aminopyrimidine-5-boronic acid pinacol ester in presence of the catalyst PdC12(dppf)DCM complex and potassium phosphate base in tetrahydrofuran solvent provides compound of formula-(IX), 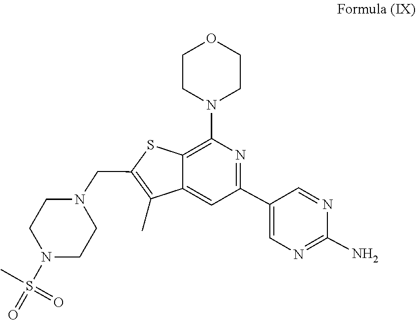 [SMILES: Cc1c(CN2CCN(S(C)(=O)=O)CC2)sc2c(N3CCOCC3)nc(-c3cnc(N)nc3)cc12]
e) treating the compound of formula-(IX) with methane sulfonic acid in aqueous methanol provides (NRC-1109) compound of formula-(II).

15. 5-[2-[(4-methylsulfonylpiperazin-1-yl)methyl]-7-morpholino-thieno [2,3-c] pyridin-5-yl]pyrimidin-2-amine) dimethane sulfonate (NRC-1111).

16. The compound as claimed in claim 15 is hydrate.

17. The crystal form of 5-[2-[(4-methylsulfonylpiperazin-1-yl)methyl]-7-morpholino-thieno [2,3-c]pyridin-5-yl]pyrimidin-2-amine) (NRC-1111 base) is characterized by:
i) Its powder X-ray diffractogram having peaks at about 9.31, 11.06, 18.64 and 20.05±0.2 degrees 2-theta.
ii) powdered X-ray diffraction pattern as shown in FIG. 1.

18. The compound as claimed in claim 15, which is a crystal form of 5-[2-[(4-methylsulfonylpiperazin-1-yl0methyl1]-7-morpholino-thieno [2,3-c]pyridine-5-yl]pyrimidin-2-amine) dimethane sulfonate hydrate (NRC-1111) and is characterized by:
i) Its powder X-ray diffractogram having peaks at about 6.19, 15.11, 18.40, 18.65, 21.60, 22.56 and 25.13±0.2 degrees 2-theta.
ii) powdered X-ray diffraction pattern as shown in FIG. 5.

19. 5-[3-methyl-2-[(4-methylsulfonylpiperazin-1-yl)methyl]-7-morpholino-thieno [2,3-c] pyridin-5-yl]pyrimidin-2-amine) dimethane sulfonate hydrate (NRC-1109).

20. The crystal form of 5-[3-methyl-2-[(4-methylsulfonylpiperazin-1-yl)methyl]-7-morpholino-thieno [2,3-c]pyridin-5-yl]pyrimidin-2-amine) (NRC-1109 base) is characterized by:
i) Its powder X-ray diffractogram having peaks at about 8.76, 9.45, 16.87, 18.39 and 19.50±0.2 degrees 2-theta.
ii) powdered X-ray diffraction pattern as shown in FIG. 3.

21. The compound as claimed in claim 19, which is a crystal form of 5-[3-methyl-2-[(4-methylsulfonylpiperazin-1-yl)methyl]-7-morpholino-thieno [2,3-c]pyridin-5-yl]pyrimidin-2-amine) dimethane sulfonate hydrate (NRC-1109) and is characterized by:
i) Its powder X-ray diffractogram having peaks at about 7.67, 10.69, 18.39, 19.58, 21.93 and 25.83±0.2 degrees 2-theta.
ii) powdered X-ray diffraction pattern as shown in figure-7.

22. The process for the preparation of crystal form of 5-[2-[(4-methylsulfonylpiperazin-1-yl)methyl]-7-morpholino-thieno [2,3-c]pyridin-5-yl]pyrimidin-2-amine) dimethane sulfonate hydrate (NRC-1111) as claimed in claim 18, comprising the steps of:
a) adding DM water to 5-[2-[(4-methylsulfonylpiperazin-1-yl)methyl]-7-morpholino-thieno [2,3-c]pyridin-5-yl]pyrimidin-2-amine (NRC-1111 base),
b) heating the reaction mixture to 90-100° C.,
c) cooling the reaction mixture and adding methanol,
d) adding methanesulfonic acid diluted in methanol to the reaction mixture,
e) stirring and filtering the mass to get the NRC-1111 dimethane sulfonate hydrate crystal form.
